(19) United States
(12) Patent Application Publication
(10) Pub. No.: US 2023/0240806 A1
(43) Pub. Date: Aug. 3, 2023

(54) ORTHODONTIC MOUTHPIECE AND ORTHODONTIC MEANS USING MOUTHPIECE

(71) Applicant: Shimoda Orthodontic Clinic, Fukuoka (JP)

(72) Inventor: Tetsuya SHIMODA, Fukuoka (JP)

(21) Appl. No.: 18/193,757

(22) Filed: Mar. 31, 2023

Related U.S. Application Data
(63) Continuation of application No. PCT/JP2020/037677, filed on Oct. 5, 2020.

(51) Int. Cl. A61C 7/08; A61C 13/34

(52) U.S. Cl. CPC A61C 7/08; A61C 13/34

(57) ABSTRACT

A conventional orthodontic mouthpiece has poor correction force compared to wire connection and hence, a large number of orthodontic mouthpieces are necessary until the completion of orthodontics and a long orthodontic period becomes necessary. Further, there has been a case where the ideal orthodontics cannot be realized with the use of only an orthodontic mouthpiece. In an orthodontic mouthpiece that performs orthodontics by covering an entire dentition that includes a tooth to be corrected with an orthodontic mouthpiece body, a portion of the orthodontic mouthpiece body that is brought into contact with a tooth in a specified dentition in the vicinity of molar teeth or in the vicinity of front teeth is cut away so as to provide a state where the specified dentition is exposed from the orthodontic mouthpiece body. Accordingly, the orthodontics can be performed using both the orthodontic mouthpiece and another tooth correcting unit.

### CROSS-REFERENCE TO RELATED APPLICATIONS

This application is a continuation application of PCT/JP2020/037677 filed on Oct. 5, 2020, which is incorporated herein by reference.

### BACKGROUND OF THE INVENTION

### Field of the Invention

The present invention relates to: an orthodontic mouthpiece that is used for performing orthodontics, and an orthodontic method that uses the mouthpiece.

### Description of the Related Art

Conventionally, for correcting teeth, the wire orthodontics has been performed using: brackets that are fixedly mounted on teeth; and a wire that is made of a shape memory alloy, is made to pass through holes formed in the brackets, and memorizes an ideal dentition mode.

However, in the wire orthodontics described above, the teeth correcting unit is visually recognized from the outside when the teeth correcting unit is worn in a mouth. Accordingly, the teeth correcting unit exhibits poor aesthetic appearance. In view of the above-mentioned circumstance, recently, a kind of orthodontics using a mouthpiece is performed where the teeth are realigned to an ideal dentition in stages using a transparent mouthpiece that exhibits excellent aesthetic appearance (see Japanese Laid-open publication 2002-526155 (patent literature 1, for example).

In the method using an orthodontic mouthpiece described in patent literature 1, the orthodontics is divided into four stages from the dentition of a patient before the orthodontics to the dentition of the patient after the orthodontics, and mouthpieces for correcting teeth are used for steadily changing the dentition until an ideal dentition is obtained. The orthodontic mouthpieces used in such a treatment are each formed of a thin sheet made of plastic or the like having flexibility.

With respect to the mouthpieces for correcting teeth that is formed as described above, the mouthpiece that approximates the dentition before orthodontics is mounted on the teeth as a orthodontic mouthpiece at a first stage.

In this case, a correcting force that intends to move the tooth toward the orthodontic mouthpiece acts on the tooth to be corrected in the dentition before orthodontics in the direction away from the orthodontic mouthpiece.

When the above-mentioned correcting force no more exists or is eliminated from the tooth to be corrected, a client is informed of the completion of correcting teeth in the first stage of the orthodontics.

Next, the orthodontic mouthpiece used in the first stage is exchanged with the orthodontic mouthpiece to be used as an orthodontic mouthpiece at a second stage of the orthodontics.

As described above, the teeth are realigned in stages by repeatedly performing the elimination and the exchange of a correcting force from the orthodontic mouthpiece, thus realizing orthodontics.

### SUMMARY OF INVENTION

However, in the conventional mouthpiece orthodontics described above, it is necessary to divide the orthodontics into four stages from the dentition of a patient before the orthodontics to the dentition of the patient after the orthodontics, and to repeat the mounting and the exchange of the orthodontic mouthpiece.

Further, in each stage, the tooth has to be moved little by little such as approximately 0.25 mm. Moreover, it is described in cited reference 1 that a period necessary for such movement of the tooth becomes is approximately two weeks.

Accordingly, the number of mouthpieces for correcting tooth is increased and hence, there is a tendency that a period necessary for completion of orthodontics is prolonged.

Further, in a case where a cheek tooth is the tooth to be corrected, since the cheek tooth has a round shape as a whole, the orthodontic mouthpiece cannot effectively transfer a force necessary for moving the cheek tooth and it is often the case that a period necessary for the completion of orthodontics is prolonged.

Orthodontics includes: the mesial movement that moves a tooth frontward in a dentition, the distal movement that moves a tooth rearward in a teeth movement, the extrusion and the intrusion that level the heights of upper surfaces of teeth, the up-lifting that moves an inclined and fallen tooth back to its proper position, and the rotation that changes the direction of a tooth, and the like.

However, the orthodontic mouthpiece that is mounted on the tooth in a covering manner is not suitable for the movement of the tooth that includes, particularly, the extrusion and the intrusion, the up-lifting and the rotation. As a result, there are some cases where orthodontics cannot be performed.

Further, when the orthodontics advances to the next stage and the orthodontic mouthpiece is exchanged in a state where the movement of some teeth to be corrected is not sufficiently performed, there is a concern that the orthodontics advances thereafter in a state that the teeth to be corrected are not being moved.

In this case, it is necessary to prepare a new mouthpiece by taking an impression of the dentition. Accordingly, a mouthpiece that is prepared in advance becomes unnecessary and hence, a material for preparing the mouthpiece is wasted.

The present invention has been made in view of the above-mentioned circumstances, and it is an object of the present invention to provide an orthodontic mouthpiece where a portion of the orthodontic mouthpiece that is brought into contact with a tooth is cut away so that the correction of teeth other than mouthpiece orthodontics can be applied to an opening portion formed by such a cut-away operation together with the mouthpiece orthodontics, and an orthodontic unit that uses the mouthpiece.

According to a first aspect of the present invention, there is provided an orthodontic mouthpiece that performs orthodontics by covering an entire dentition that includes a tooth to be corrected with an orthodontic mouthpiece body, wherein a portion of the orthodontic mouthpiece that is brought into contact with a tooth of the orthodontic mouthpiece body that corresponds to a specified dentition in a vicinity of molars or in a vicinity of front teeth is cut away so as to provide a state where the specified dentition is exposed from the orthodontic mouthpiece body.

According to a second aspect of the present invention, there is provided a orthodontic method that uses the orthodontic mouthpiece according to claim 1 in correcting the dentition by covering an entire dentition that includes a tooth to be corrected with the orthodontic mouthpiece body, the orthodontic method comprising the steps of: taking a model of a pre-orthodontic dentition of a patient using a plaster or an intraoral scanner; separating by cutting the model prepared from the mold of the dentition or scanner data for respective teeth and preparing single models for individual teeth; realigning the single models for individual teeth to an ideal dentition after correction; inserting a correcting wire for correcting the specified dentition into a wire jig that is fixedly connected to a target portion of each tooth of the specified dentition thus providing a shared correcting wire for correcting the specified dentition; removing a correcting wire for correcting the specified dentition and an ideal correcting wire from the respective teeth of the specified dentition and mounting the wire jig and the shared correcting wire on the specified dentition of the patient; fixedly mounting the wire jig to the target portions of the specified dentition, inserting the shared correcting wire in the jig, and fixing front and rear ends of the shared correcting wire to the dentition in mounting the wire jig and the shared correcting wire on the dentition of the patient; fixing, by covering, a mouthpiece where a portion of the mouthpiece that is brought into contact with a tooth to be corrected and a tooth in a vicinity of the tooth to be corrected in the specified dentition is cut away; correcting the dentition to a predetermined dentition while performing a tightening operation of the shared correcting wire and an exchange of the mouthpiece along with a progress of the orthodontics; and removing the mouthpiece, the shared correcting wire and the wire jig after completion of the orthodontics.

According to the orthodontic mouthpiece of the first aspect of the present invention, by cutting away a portion of the orthodontic mouthpiece body that corresponds to the specified dentition in the vicinity of the molar teeth or the front teeth and is brought into contact with the teeth, it is possible to acquire an advantageous effect that orthodontics can be applied to the exposed target portion using other correcting unit.

Further, according to the second aspect of the present invention, by mounting the wire jig and the correcting wire to the dentition of the patient and, thereafter, by mounting the orthodontic mouthpiece where a portion of the orthodontic mouthpiece body that is brought into contact with the tooth to be corrected and the tooth in the vicinity of the tooth to be corrected is cut away on the dentition of the patient, the wire correction having a high correcting force is applied to a portion at which the mouthpiece orthodontics is not suitable so that the required number of the orthodontic mouthpieces can be reduced whereby a correcting period can be shortened.

By using the orthodontic mouthpiece where a portion such as an occlusal surface or a tongue side surface that is not easily visually recognizable together with the wire jig, the dentition can acquire high aesthetic appearance.

### BRIEF DESCRIPTION OF THE DRAWINGS

FIG. 1 is a schematic perspective view illustrating a state where orthodontics is performed using an orthodontic mouthpiece according to a first embodiment of the present invention;

FIG. 2 is a schematic perspective view illustrating a mouthpiece single unit for orthodontics according to the first embodiment of the present invention;

FIG. 3 is a schematic enlarged plan view of a tooth surface cutaway portion of the orthodontic mouthpiece according to the first embodiment of the present invention;

FIG. 4 is composed of a schematic plan view and a cross-sectional view illustrating the orthodontic mouthpiece in a state where the mouthpiece orthodontics is in a state where orthodontics has started;

FIG. 5 is composed of a schematic plan view and a cross-sectional view illustrating the orthodontic mouthpiece in a state where the mouthpiece orthodontics is in a state where orthodontics is being performed;

FIG. 6 is a flowchart illustrating an orthodontic method using the orthodontic mouthpiece of the present invention;

FIG. 7 is a schematic perspective view illustrating a wire jig used in the orthodontic method of the present invention; and

FIG. 8 is a schematic perspective view illustrating another wire jig used in the orthodontic method of the present invention.

### DETAILED DESCRIPTION OF THE PREFERRED EMBODIMENTS

Hereinafter, an embodiment of an orthodontic mouthpiece and an orthodontic method using the orthodontic mouthpiece is described in detail with reference to drawings. In the drawings, a cutaway portion of the orthodontic mouthpiece is formed on an occlusal surface. However, the cutaway portion may be formed on a tongue side or on a cheek side.

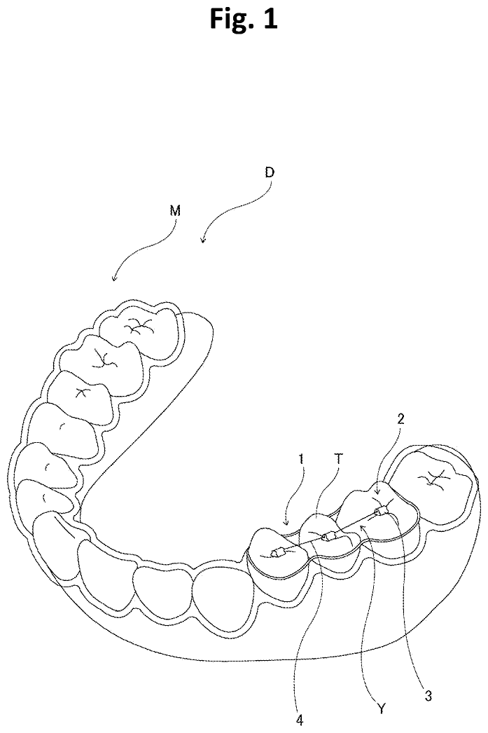

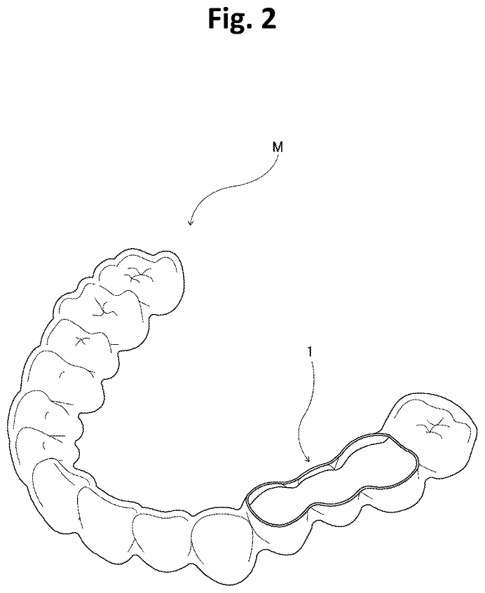

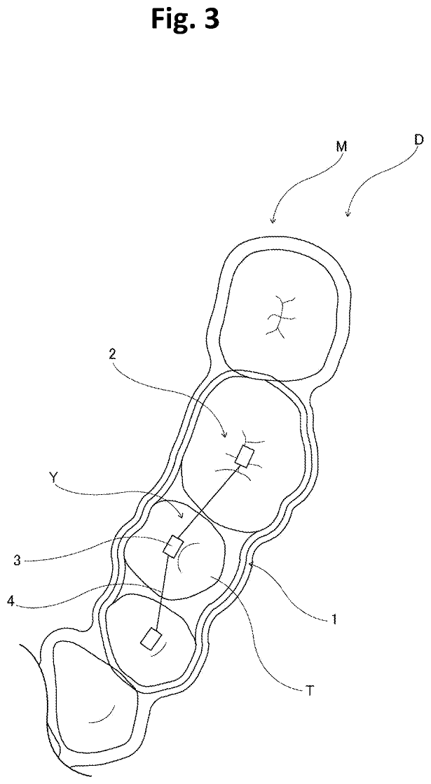

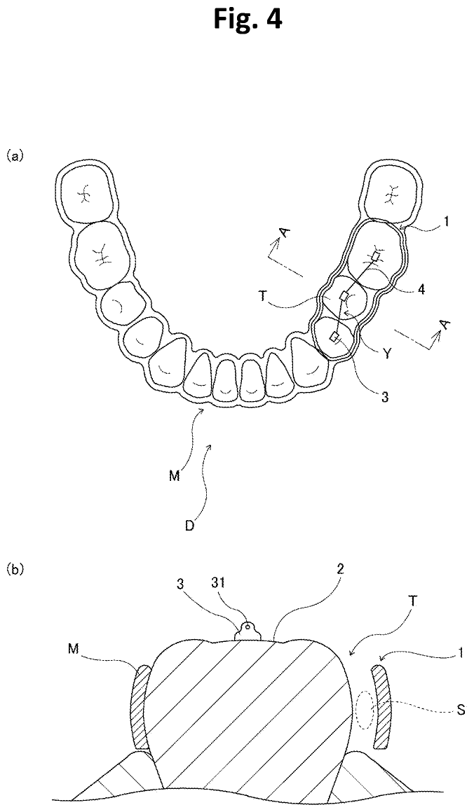

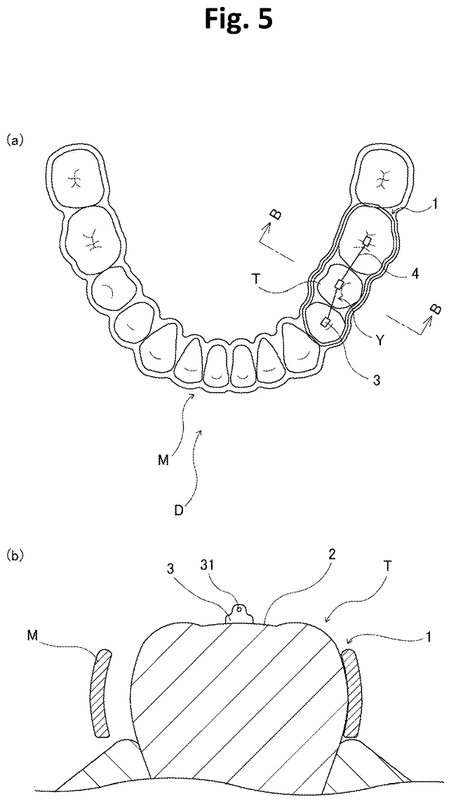

FIG. 1 is a schematic perspective view illustrating a state where orthodontics is performed using the orthodontic mouthpiece according to the present invention. FIG. 2 is a schematic perspective view illustrating a mouthpiece single unit for orthodontics according to the present invention. FIG. 3 is a schematic enlarged plan view of a tooth surface cutaway portion of the orthodontic mouthpiece according to the present invention. FIG. 4 is composed of a schematic plan view and a cross-sectional view illustrating the orthodontic mouthpiece in a state where the mouthpiece orthodontics is in a state where orthodontics has started. FIG. 5 is composed of a schematic plan view and a cross-sectional view illustrating the orthodontic mouthpiece in a state where the mouthpiece orthodontics is in a state where orthodontics is being performed.

### 1. Orthodontic Mouthpiece

As illustrated in FIG. 1, an orthodontic mouthpiece M according to the present invention is mounted in a covering state on a dentition D of either of or both of an upper jaw and a lower jaw that includes a tooth to be corrected (hereinafter the description being made based on the dentition on the lower jaw).

A tooth surface cutaway portion 1 is formed in the orthodontic mouthpiece M in the vicinity of back teeth.

The tooth surface cutaway portion 1 is formed so as to expose a portion that is brought into contact with the teeth, that is, any one of an occlusal surface 2, a tongue side surface or a cheek side surface at a molar portion of the dentition D, and is formed so as to expose a tongue side surface or a cheek side surface at an incisor of the dentition D.

The orthodontic mouthpiece M is formed of a thin plastic sheet having resiliency.

In a case where a thickness of the plastic sheet is less than 0.2 mm, it is difficult to form the orthodontic mouthpiece M, and the orthodontic mouthpiece M exhibits poor durability so that the orthodontic mouthpiece M is easily broken. On the other hand, In a case where the thickness of the plastic sheet is 1 mm or more, a patient feels discomfort at the time of mounting the orthodontic mouthpiece M in his mouth.

Accordingly, it is preferable that the plastic sheet be formed with a thickness of approximately from 0.2 to 1 mm inclusive.

A portion of the mouthpiece M where the tooth surface cutaway portion 1 is formed at a molar portion or at an incisor portion to which a force necessary for orthodontics cannot be effectively transferred to a tooth to be corrected in mouthpiece orthodontics. The “portion of the mouthpiece M where the tooth surface cutaway portion 1 is formed” is hereinafter also referred to as “target portion”.

With respect to the portion of the mouthpiece M where the tooth surface cutaway portion 1 formed, in forming the orthodontic mouthpiece M, a space S (see FIG. 4(a)) having a size of approximately 1 to 2 mm from a cheek side to a tongue side of a tooth T to be corrected before the orthodontics is formed.

With respect to portions of the mouthpiece M where the cutaway portion is not formed, these portions are formed in the same manner as a conventional mouthpiece is formed.

With the configuration formed as described above, when the orthodontic mouthpiece M is mounted on the dentition D in a covering manner, a portion that is brought into contact with the teeth at the target portion, that is, any one of the occlusal surface 2, the tongue side surface and the cheek side surface is exposed from the tooth surface cutaway portion 1.

Then, the orthodontic mouthpiece M engages with the dentition D by portions other than the tooth surface cutaway portion 1 by fitting and can be held by these portions.

Further, by exposing any one of the occlusal surface 2, the tongue side surface and the cheek side surface of the dentition D from the opening formed by the tooth surface cutaway portion 1, a wire correcting unit Y described later can be disposed on the exposed target portion. Accordingly, it is possible to prevent the physical interference between the orthodontic mouthpiece M and the wire correcting unit Y.

As a result, it is possible to perform the orthodontics using the orthodontic mouthpiece M having the high aesthetic appearance without obstructing the orthodontics by the wire correcting unit Y.

Further, during the mouthpiece orthodontics, it is necessary for a patient himself/herself to remove the orthodontic mouthpiece M so as to have a meal or to take care of the inside of his/her mouth such as teeth brushing.

Also in this respect, the formation of the tooth surface cutaway portion 1 in the orthodontic mouthpiece M that avoids the interference between the orthodontic mouthpiece M and the wire correcting unit Y allows the patient to easily remove the orthodontic mouthpiece M.

Next, the wire correcting unit Y that is mounted in the tooth surface cutaway portion 1 of the orthodontic mouthpiece M is described in detail.

As illustrated in FIG. 3 to FIG. 5, the wire correcting unit Y includes: the wire jigs 3 that are fixedly mounted on the target portion of the dentition D, that is, any one of the occlusal surface 2, the tongue side surface and the cheek side surface of the tooth; and a correcting wire 4 that is made to pass through the wire jigs 3.

The wire jigs 3 are, as illustrated in FIG. 3 to FIG. 5, formed by applying by coating a resin having a mountain-like shape and a large thickness to the target portions of the teeth T to be corrected on the dentition model prepared based on the actual dentition of the patient.

The wire jig 3 is formed so as to have an uneven shape corresponding to an uneven shape of the surface of any one of the occlusal surface 2, the tongue side surface and the cheek side surface of the tooth to be corrected of the individual patient, by curing the resin having a large thickness applied to the dentition model as described above.

After the above-mentioned wire jigs 3 are formed, the wire jigs 3 are fixedly mounted on the target portions of the teeth T to be corrected of the patient using a dental adhesive agent.

The correcting wire 4 is a wire made of a shape memory alloy, stainless steel or the like.

The correcting wire 4 is made to pass through the through holes 31 formed in the wire jigs 3.

The above-mentioned through holes 31 are formed by embedding in advance the correcting wire 4 in which the shape is memorized such that the shape follows the dentition in an ideal state in a resin that is still uncured at the time of forming the wire jigs 3 described above.

Further, corresponding to a progress state of the orthodontics, the correcting wire 4 having a diameter smaller than a diameter of the through hole 31 is used as a starting correcting wire 4 and, thereafter, the starting correcting wire 4 is exchanged with the succeeding correcting wires 4 that gradually increase their diameters.

When the wire correcting unit Y is mounted on the occlusal surface 2 of the tooth T to be corrected, the wire correcting unit Y obstructs mystifications of a patient.

When the mystifications of the patient are obstructed, a masticatory muscle of the patient is weakened so that the tightened gingiva is loosened and hence, the progress of orthodontics can be accelerated.

Further, with the use of the wire correcting unit Y, the correction is performed by moving the tooth using a restoring force of the wire. In this case, there has been a case where the tooth is moved in an unintended direction or in a largely displaced manner beyond the estimation.

Particularly, there is a tendency that such a drawback is likely to occur when partial correction is performed.

However, as illustrated in FIG. 4 and FIG. 5, the space S that is provided in the formation of the orthodontic mouthpiece M limits a moving range of the tooth. That is, the tooth T to be corrected impinges on either one of a cheek side surface 11 and a tongue side surface 12 of the mouthpiece M that form the space S and hence, the cheek side surface 11 and the tongue side surface 12 function as stoppers and can prevent the movement of the tooth T to be corrected beyond a limited distance.

Further, a state where the tooth T to be corrected impinges on either one of the cheek side surface 11 and the tongue side surface 12 also plays a role of a guide that informs a patient of a timing of exchanging the correcting wire 4 and a timing of exchanging the orthodontic mouthpiece M.

That is, the state where one of the cheek side surface 11 and the tongue side surface 12 impinges on the teeth T to be corrected is a state where the tooth moves to the position that the wire correcting unit Y aims at so that the orthodontics is completed. Accordingly, the state plays a milestone that allows the operation to advance to a next step.

### 2. Steps Related to Orthodontic Method

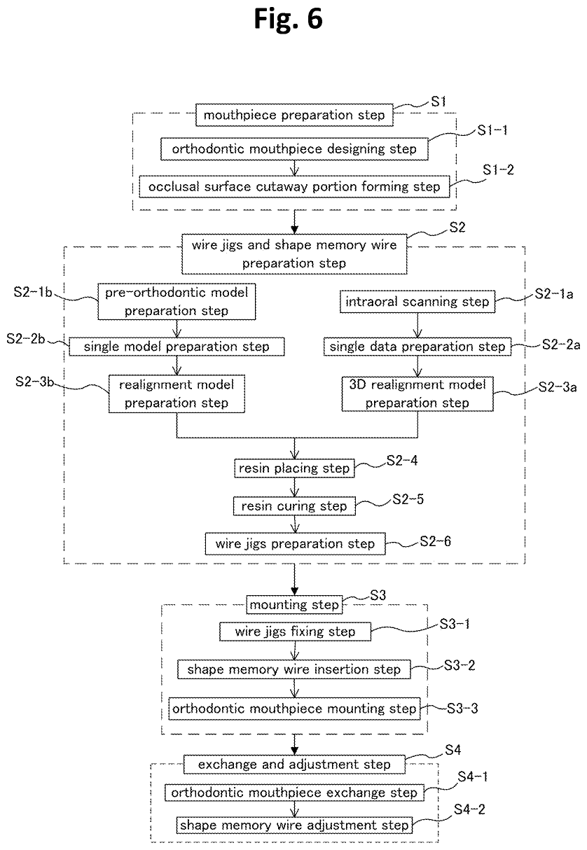

Next, the orthodontic method is described in detail with reference to drawings. FIG. 6 is a flowchart illustrating steps related to the orthodontic method that uses the orthodontic mouthpiece according to the present invention.

That is, as illustrated in the drawing, the orthodontic method A that uses the orthodontic mouthpiece according to the present invention includes: a step S1 of preparing an orthodontic mouthpiece M; a step S2 of preparing the wire jig and the correcting wire; a step S3 of mounting the orthodontic mouthpiece M, the wire jig and the connecting wire on a patient; and an exchange and adjustment step S4 of performing an exchange of the orthodontic mouthpiece M and the adjustment of the wire correcting unit.

The step S1 of preparing the orthodontic mouthpiece M is a step where a course of orthodontics for each individual patient is determined, and an orthodontic mouthpiece M that is suitable for the course is prepared.

The step S1 of preparing the orthodontic mouthpiece M includes a step S1-1 of designing the orthodontic mouthpiece M, and a step S1-2 of forming a tooth surface cutaway portion.

In the step S11 of designing the orthodontic mouthpiece M, firstly, the movement of the tooth is divided into a plurality of stages from the dentition of a patient before orthodontics to the dentition of the patient after the dentition, and the simulation of the movement of the actual tooth is performed.

In the simulation, the teeth to be corrected in the dentition that cannot be moved by orthodontics using only the orthodontic mouthpiece M, and the teeth to be connected in the dentition where the number of required orthodontic mouthpiece M is increased so that a period for orthodontics is prolonged are determined.

In the above-mentioned teeth to be corrected determine as described above, wire correcting unit Y is used together with the orthodontic mouthpiece M. Accordingly, the space S is formed on the cheek side or on the tongue side of the tooth T to be corrected by an amount corresponding to the movement of the tooth that is estimated when the wire correcting is performed in each stage. To be more specific, the space S of 1 to 2 mm is formed on the cheek side or on the tongue side of the tooth T.

The determination portion is a molar portion that the orthodontic mouthpiece M cannot effectively correct or an incisor portion that is determined to require a long period for orthodontics. The portion is the dentition D positioned on both of or either one of the upper and lower jaws. The portion may be also both of or either one of the left and right molar portions, or may be only the incisor portion or the combination of the incisor portion and the molar portion.

The simulations described heretofore are performed on a computer so as to design the orthodontic mouthpiece M.

Then, a plastic sheet is actually formed based on the design, and prepares the individual orthodontic mouthpiece M that is required for orthodontics.

The next step is the tooth surface cutaway portion forming step S1-2 of forming the portions of the orthodontic mouthpiece M where the wire correcting unit Y is used in a state where the target portion of the tooth T to be corrected, that is, the either one of the occlusal surface 2, the tongue side surface and the cheek side surface is exposed.

This tooth surface cutaway portion forming step S1-2 is a step of forming a cutaway opening in the position where the wire correcting unit Y is used in the orthodontic mouthpiece M in step S1-1 of designing the orthodontic mouthpiece M so as to prevent the orthodontic mouthpiece M from physically interfering with the wire correcting unit Y.

In forming the cutaway opening, the operation is performed such that the target portion of the tooth T to be corrected of a patient is exposed when the orthodontic mouthpiece M is mounted.

The wire correcting unit Y applied to such a target portion does not physically interfere with the orthodontic mouthpiece M by forming the opening in the orthodontic mouthpiece M. Accordingly, the movement of the tooth T to be corrected is not obstructed by the wire correcting unit Y.

Further, the patient can remove the mouthpiece M when necessary at the time of having a meal or at the time of brushing teeth so that the patient can make the inside of his/her mouth clean.

A wire jig and correcting wire manufacturing step S2 is constituted of the following six steps.

A pre-orthodontic dentition model preparation step S2-1a of preparing a dentition model before correcting.

A single tooth model preparation step S2-2a of preparing individual single teeth models from the pre-orthodontic dentition model.

A realignment model preparation step S2-3a of realigning the single teeth models into an ideal dentition.

A resin applying step S2-4 of applying a resin that is a material for forming the wire jig 3 to any one of an occlusal surface 2, a tongue side surface or a cheek side surface of the realignment model.

A resin curing step S2-5 of embedding a correcting wire 4 that is bent in accordance with an ideal dentition, and curing the resin by emitting a UV light to the resin

A wire jig preparation step S2-6 of forming the wire jig 3 by removing the cured resin and the correcting wire 4 from the realignment model

The pre-orthodontic dentition model preparation step S2-1a is a step where a dentition cast of the patient is taken using alginate or the like and, thereafter, a plaster for molding is filled into the dentition cast thus forming pre-orthodontic dentition model.

The single model preparation step S2-2a is a step where the single teeth models are cut out from each other one by one from the prepared pre-orthodontic dentition model thus preparing the individually independent single teeth models.

The realignment model preparation step S2-3a is a step where the single models are arranged on a base, and are rearranged to form the ideal dentition.

The pre-orthodontic dentition model preparation step S2-1a, the single model preparation step S2-2a, and the realignment model preparation step S2-3a described above are all steps performed using an analogue technique. However, the steps may be also performed by a digital technique.

That is, as described in FIG. 6, the respective steps may be replaced with an intraoral scanning step S2-1b, a single data preparation step S2-2b, and a 3D realignment model preparation step S2-3b.

The intraoral scanning step S2-1b is a step where a dentition state of a patient before correcting is scanned by an intraoral scanner, and 3D data of pre-orthodontic dentition is supplied to a computer.

The single data preparation step S2-2b is a step where 3D data of the pre-orthodontic dentition obtained in the preceding step is divided so as to prepare individual single data on the computer.

The 3D realignment model preparation step S2-3b is a step where the individual single data that are prepared in the preceding step are rearranged on the computer so as to prepare data of an ideal dentition state, and a model is prepared using a 3D printer.

As the step of preparing the model using the 3D printer, a suitable method can be selected based on the determination made by a dentist. Any method can be used provided that a model in an ideal dentition state that is used in the next step can be prepared.

The resin applying step S2-4 is a step where a resin that is used as a material for forming the wire jig 3 is applied by coating to any one of the occlusal surface 2′, the tongue side surface and the cheek side surface corresponding to the specified dentition that is scheduled to use the wire correcting unit Y formed of the realignment model prepared in the preceding step.

In this step, the resin is applied by coating to any one of the occlusal surface 2′, the tongue side surface and the cheek side surface with a large thickness in a mountain shape.

In the resin curing step S2-5, a correcting wire 4 that memorizes a shape following an ideal dentition is embedded in the resin that is applied by coating to any one of the occlusal surface 2′, the tongue side surface and the cheek side surface of the realignment model in the preceding step, and the resin is cured by emitting a UV light to the resin.

The wire jig preparation step S2-6 is a step where the embedded correcting wire 4 is pulled out from the cured resin on the realignment model, and the resin is removed from the model.

Further, it is needless to say that, to facilitate pulling out of the embedded correcting wire 4 from the cured resin, a petroleum jelly or the like may be applied by coating to a surface of the correcting wire 4 at the time of embedding the correcting wire 4 into an uncured resin.

A hole that is formed when the correcting wire 4 is pulled out from the wire jig 3 that is formed by curing the resin becomes a through hole 31.

The mounting step S3 includes a wire jig fixing step S3-1, a correcting wire insertion step S3-2, and an orthodontic mouthpiece mounting step S3-3.

The wire jig fixing step S3-1 is a step where the wire jig 3 that is prepared in the wire jig preparation step S2-6 is fixedly mounted on actual teeth of a patient.

To be more specific, the wire jig 3 that corresponds to the target portion of the teeth T to be corrected by using the wire correcting unit Y, that is, any one of the occlusal surface 2, the tongue side surface and the cheek side surface of the teeth T to be corrected is fixedly mounted by a dental adhesive.

The wire jig 3 is prepared based on the dentition model of the patient. Accordingly, the wire jig 3 has a shape that corresponds to a surface uneven shape of any one of the occlusal surface 2′, the tongue side surface and the cheek side surface that uses the wire correcting unit Y and hence, the wire jig 3 can be fixedly mounted at the accurate position with an accurate angle.

The correcting wire insertion step S3-2 is a step where the correcting wire 4 is inserted into and is fixed to the through hole 31 that is formed by embedding the correcting wire 4 at the time of forming the wire jig 3.

In this step, the through hole 31 of the wire jig 3 is formed using the realignment model (ideal dentition after orthodontics). As a result, when the wire jig 3 is fixedly mounted on the dentition before correcting, the directions of the holes become non-uniform.

Accordingly, as the correcting wire 4 to be inserted into the through hole 31, a correcting wire having a small diameter that is bent with the same curve as a correcting wire that is used when the wire jig 3 is formed is inserted and, in an exchange and adjustment step S4 that is a next step, the correcting wire is exchanged with a correcting wire having a larger diameter as the stage advances from the preceding stage to the succeeding stage.

The orthodontic mouthpiece mounting step S3-3 is a step where, after the wire jig 3 and the correcting wire 4 are mounted on the dentition of the patient, the orthodontic mouthpiece M that is prepared in the orthodontics mouthpiece manufacturing step S1 is mounted on the entire dentition of the patient in a covering manner.

The orthodontic mouthpiece M is mounted on the entire dentition D in a covering manner such that the target portions that use the wire correcting unit Y, that is, the occlusal surface 2, the tongue side surface or the cheek side surface of the orthodontic mouthpiece M is exposed by the formed tooth cutaway portion 1.

That is, the orthodontic mouthpiece M and the wire correcting unit Y are mounted on the dentition D of the patient without causing the physical interference between the orthodontic mouthpiece M and the wire correcting unit Y.

The exchange and adjustment step S4 includes: an orthodontics mouthpiece exchange step S4-1 for exchanging the orthodontic mouthpiece M in each correcting stage; and a correcting wire adjustment step S4-2 for adjusting the correcting wire 4 used in the wire correcting unit Y.

The orthodontics mouthpiece M exchange step S4-1 is a step where, when a correcting force that is applied to the tooth T to be corrected is eliminated, the orthodontic mouthpiece M is exchanged with the orthodontic mouthpiece M in the next correcting stage.

The orthodontics mouthpiece M exchange step S4-1 is performed at the same timing until the completion of correcting, and the orthodontic mouthpiece M is exchanged with the orthodontic mouthpiece M in the next correcting stage suitably such that a state where a force relating to correcting is basically applied to the teeth until the completion is maintained.

The correcting wire 4 adjustment step S4-2 is a step where the correcting wire 4 is exchanged at a timing that the correcting wire 4 that is inserted into the wire jigs 3 takes the memorized original curve.

Since the wire jigs 3 are formed based on the realignment model, the formed wire jigs 3 are disposed in non-uniform directions from each other when the wire jigs 3 are fixedly mounted on the dentition D before correcting. In such a step, as a matter of course, the through holes 31 formed in the wire jig 3 are directed in non-uniform directions from each other respectively.

By inserting the correcting wire 4 into the through holes 31, a state is brought about where the correcting wire W is inserted into the through holes 31 in a zigzag shape.

The wire correcting unit Y corrects the teeth to be corrected by making use of a force that makes the correcting wire 4 to return to the original curve.

That is, when the correcting wire 4 returns to the original curve so that patient feels no pressure from the wires jigs 3 on the teeth T to be corrected, in the correcting wire 4 adjusting step S4-2, the inserted correcting wire is exchanged with the new correcting wire.

With respect to the exchange of the correcting wire 4, by setting a diameter of the exchanging correcting wire 4 larger than a diameter of the correcting wire 4 before the exchange, correcting of the teeth is performed toward an ideal teeth state in stages.

By exchanging the correcting wire 4 having a smaller diameter from the correcting wire 4 having a larger diameter in this manner, the correcting wire 4 is finally exchanged to the correcting wire having the same diameter as the correcting wire 4 that is used at the time of forming the wire jig 3. When a pressure that intends to return the correcting wire 4 to the original shape is no more applied to the teeth to be corrected, the orthodontics is completed.

Further, in such partial correcting wire, although it is a rare case, there is a possibility that the correcting wire 4 is formed into an unexpected shape so that the tooth is moved excessively.

To prevent the occurrence of such a case, as illustrated in FIG. 4 and FIG. 5, a cheek side surface 11 and a tongue side surface 12 that remain on the teeth surface cutout portion 1 of the orthodontic mouthpiece M restrict the movement of the teeth like walls thus functioning as stoppers that prevent the excessive movement of the teeth.

Further, the timing that the teeth impinge on the cheek surface 11 or the tongue side surface 12 becomes a benchmark that the further wire correcting is not performed. As a result, the cheek side surface 11 and the tongue side surface 12 also play the role of guides by which the patient knows the timing of the next wire adjustment.

### 3. Other Embodiments

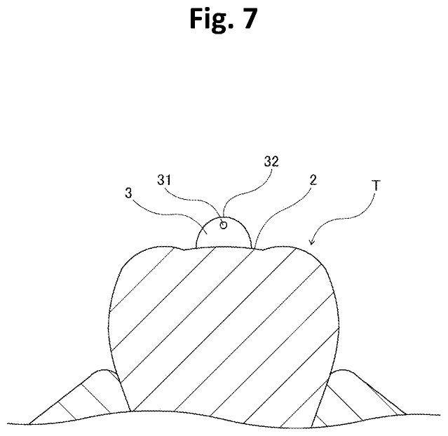

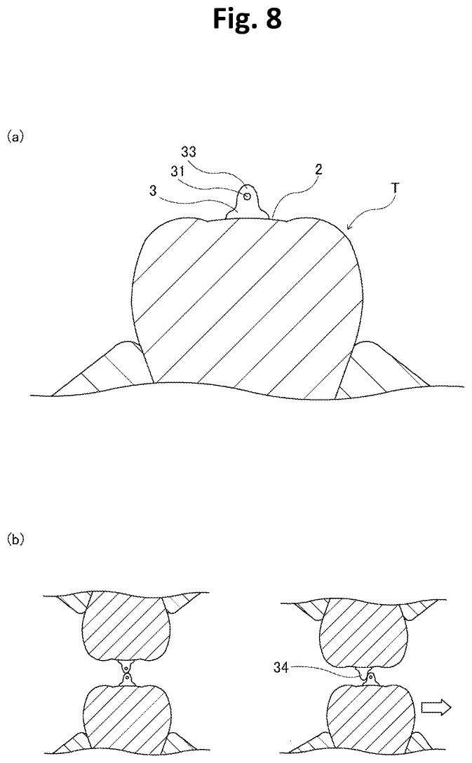

Next, other embodiments of the wire jig 3 according to the present invention are described in detail with reference to drawings. FIG. 7 and FIG. 8 are schematic perspective views illustrating other examples of the wire jig 3 according to an orthodontic method of the present invention.

As other embodiment of the wire jig 3, as illustrated in FIG. 7, the wire jig 3 has a shape where an upper surface of the wire jig. 3 has a smooth upper surface.

This shape is formed by grinding and forming a resin removed from a realignment model in the wire jig 3 forming step S2-6 described above.

By forming the upper surface into a smooth curved surface 32, upper and lower gingiva slide from each other at the time of biting and hence, it is possible to acquire an advantageous effect of obstructing chewing more effectively.

That is, the wire jig 3 has the shape that further increases an effect of loosening the gingiva by weakening a chewing muscle described above.

With respect to this embodiment, a case is considered where the upper and lower gingiva slide from each other at the time of biting. Accordingly, a case is considered where the embodiment is applied to a patient having a habit of involuntary nocturnal tooth grinding, clenching or the liked during sleeping.

Further, in another embodiment described in FIG. 8a and FIG. 8b, the wire jig 3 has the configuration where an arbitrary protruding portion 33 is formed on the wire jig 3.

Also such a shape is formed by grinding and forming a resin removed from a realignment model in the wire jig 3 forming step S2-6 described above.

As illustrated in FIG. 8b, for example, by forming the arbitrary protruding portion 33 in an upwardly protruding manner, with respect to an occlusal surface of a tooth that corresponds to chewing, that is, the gingiva on a right side of the lower jaw, the protruding portion 33 is brought into contact with an uneven surface of the occlusal surface of the gingiva on the right side of the upper jaw. Accordingly, the protruding portion 33 is brought into contact with an uneven shape of the occlusal surface of the gingiva on the right side of the upper jaw and hence, the wire jig 3 is pushed so that a force is added in a moving direction of the tooth to be corrected T whereby it is possible to acquire an advantageous effect that a correcting force is increased.

It is considered that this embodiment is used in a case where the correction requires a particularly long time even when a wire correcting unit Y is used.

Further, in using another embodiment described above, a correcting auxiliary jig 34 formed by curing a resin may be applied to a tooth that does not require the correcting corresponding to the biting with respect to the tooth T to be corrected to which the wire jig 3 is fixedly mounted.

The correcting auxiliary jig 34 is provided for making the advantageous effect acquired by the above- mentioned biting more effective, and may be used when necessary.

Further, in any one of the above-mentioned embodiments, the wire jig 3 having a certain shape may be always used during the correcting period. However, the wire jig 3 may be exchanged with a wire jig 3 that can be used more efficiently based on the determination made by a dentist during a correcting period.

The orthodontic method using the orthodontic mouthpiece M according to the present invention is, as described heretofore, characterized by realizing both the more efficient orthodontics and high aesthetic appearance by jointly using the correcting by the orthodontic mouthpiece M and the wire correcting unit Y.

However, the orthodontics may not be always limited to a case where the orthodontics is performed using only the orthodontic mouthpiece M. Orthodontics may be performed for the purpose of acquiring advantageous effects obtained by a stopper or a guide formed by the space S in the teeth surface cutaway portion 1 formed in the orthodontic mouthpiece M.

In this case, the teeth surface cutaway portion 1 of the orthodontic mouthpiece M is prepared as described above, other portions do not require correcting force and hence, other portions may be prepared in a shape that conforms to the dentition of the patient.

The present invention has been described with reference to the above-mentioned embodiments heretofore, the present invention is not limited to such embodiments. Further, the above-mentioned respective advantageous effects merely enumerate the most appropriate advantageous effects acquired by the present invention, and the advantageous effects of the present invention are not limited to the advantageous effects described in the embodiments.

What is claimed is:

1. An orthodontic mouthpiece that performs orthodontics by covering an entire dentition that includes a tooth to be corrected with an orthodontic mouthpiece body, wherein
a portion of the orthodontic mouthpiece that is brought into contact with a tooth of the orthodontic mouthpiece body that corresponds to a specified dentition in a vicinity of molars or in a vicinity of front teeth is cut away so as to provide a state where the specified dentition is exposed from the orthodontic mouthpiece body.

2. A orthodontic method that uses the orthodontic mouthpiece according to claim 1 in correcting the dentition by covering an entire dentition that includes a tooth to be corrected with the orthodontic mouthpiece body, the orthodontic method comprising the steps of:
taking a model of a pre-orthodontic dentition of a patient using a plaster or an intraoral scanner;
separating by cutting the model prepared from the mold of the dentition or scanner data for respective teeth and preparing single models for individual teeth;
realigning the single models for individual teeth to an ideal dentition after correction;
inserting a correcting wire for correcting the specified dentition into a wire jig that is fixedly connected to a target portion of each tooth of the specified dentition thus providing a shared correcting wire for correcting the specified dentition;
removing a correcting wire for correcting the specified dentition and an ideal correcting wire from the respective teeth of the specified dentition and mounting the wire jig and the shared correcting wire on the specified dentition of the patient;
fixedly mounting the wire jig to the target portions of the specified dentition, inserting the shared correcting wire in the jig, and fixing front and rear ends of the shared correcting wire to the dentition in mounting the wire jig and the shared correcting wire on the dentition of the patient;
fixing, by covering, a mouthpiece where a portion of the mouthpiece that is brought into contact with a tooth to be corrected and a tooth in a vicinity of the tooth to be corrected in the specified dentition is cut away;
correcting the dentition to a predetermined dentition while performing a tightening operation of the shared correcting wire and an exchange of the mouthpiece along with a progress of the orthodontics; and
removing the mouthpiece, the shared correcting wire and the wire jig after completion of the orthodontics.
